(19) United States
(12) Patent Application Publication
(10) Pub. No.: US 2026/0224817 A1
(43) Pub. Date: Aug. 6, 2026

(54) SYRINGE WITH MIXING ELEMENT

(71) Applicant: SHAARE ZEDEK SCIENTIFIC LTD., Jerusalem (IL)

(72) Inventors: Yoav GRONOVICH, Maale Hahamisha (IL); Yehuda BACHAR, Yehud-Monosson (IL)

(21) Appl. No.: 19/111,621

(22) PCT Filed: Sep. 13, 2023
(86) PCT No.: PCT/IL2023/050996

§ 371 Date: Mar. 13, 2025

(51) Int. Cl. A61M 5/315; A61M 5/19; A61M 5/31

(52) U.S. Cl. CPC A61M 5/31596; A61M 5/19; A61M 5/3129; A61M 5/31511; A61M 2207/00

(57) ABSTRACT

Provided is a syringe, including a barrel having an outer wall with an essentially constant diameter, and an inner wall defining at least two compartments and at least one channel interconnecting the at least two compartments, wherein the diameter of each of the at least two compartments is larger than a diameter of the channel, and a plunger sized to fit within the channel wherein when the plunger is positioned within the channel the at least two compartments are sealed off from one another, thereby preventing mixing of contents contained in each of the at least two compartments, and such that withdrawal of the plunger from the channel allows mixing of the contents contained in each of the at least two compartments.

### FIELD OF THE INVENTION

The present invention, in some embodiments thereof, relates to a syringe with a mixing element.

### BACKGROUND

Botulinum toxin and hyaluronic acid injections are the most common aesthetic procedures worldwide. Today, some new approaches, such as Mesobotox and Microbotox, use a combination of hyaluronic acid (HA) and botulinum toxin for a wide range of aesthetic treatments, such as in use in the forehead, periorbital area, cheeks, lips and perioral area, chin, neck, dorsal hands, etc. These approaches use the ability of the botulinum to denervate the muscles and reduce the sweat and sebaceous glands activity, as well as the ability of the HA to improve the skin quality and tone, in a synergistic effect. In common practice, mixing of botulinum and HA is done manually by using two different syringes that are connected to each other, which may be uncomfortable, time consuming, and hard to be kept sterile.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

According to some embodiments, there is provided a multiple compartment syringe configured to store a plurality of contents separately, such that the different contents are sealed off from one another within different compartments.

According to some embodiments, the multiple compartment syringe may be configured to mix a plurality of contents therein, thereby preventing use of more than one syringe in a procedure, such as for example, by combining hyaluronic acid and botulinum toxin within the syringe instead of applying the hyaluronic acid and botulinum toxin with separate needles, for procedures such as Mesobotox and Microbotox.

According to some embodiments, the multiple compartment syringe may be configured to enable mixing of the plurality of contents stored therein by withdrawal of the plunger. According to some embodiments, the multiple compartment syringe may be configured to enable mixing two contents while maintaining a third content sealed off from the mixed first and second contents. According to some embodiments, the multiple compartment syringe may be configured to enable mixing of the contents without injecting the mix through a needle hub thereof, thereby allowing the contents to mix over a desired period of time.

According to some embodiments, there is provided a multiple compartment syringe, including: a barrel including an outer wall having an essentially constant diameter and an inner wall defining at least two compartments and at least one channel interconnecting the at least two compartments, wherein the diameter of each of the at least two compartments is larger than a diameter of the channel, and a plunger sized to fit within the channel wherein when the plunger is positioned within the channel the at least two compartments are sealed off from one another, thereby preventing mixing of contents contained in each of the at least two compartments, and wherein withdrawal of the plunger from the channel allows mixing of the contents contained in each of the at least two compartments.

According to some embodiments, the inner wall at the circumference of the channel is configured to sealingly abut the plunger. According to some embodiments, the plunger includes a sealing material. According to some embodiments, at least one compartment is a mixing compartment configured to receive contents from a first compartment and a second compartments at an open configuration of the multiple compartment syringe in which the plunger has been at least partially withdrawn, thereby enabling mixing of two or more contents of two or more compartments.

According to some embodiments, the plunger includes a mixing mechanism configured to stir contents within the one or more compartments at the open configuration of the multiple compartment syringe. According to some embodiments, the plunger includes a content insertion mechanism configured to enable insertion of content into a compartment at a closed configuration of the multiple compartment syringe in which the plunger is positioned within the channel.

According to some embodiments, the plunger includes a lumen and a sealable opening, the lumen enabling insertion of content through the sealable opening into at least one compartment of the barrel. According to some embodiments, the sealable opening is moveable in relation to the barrel such that at an open position of the sealable opening, the lumen of the plunger is in fluid communication with one of the compartments of the plurality of compartments of the barrel.

According to some embodiments, the inner wall of the barrel includes at least one divider positioned within the lumen of the barrel and configured to prevent fluid communication between two or more compartments. According to some embodiments, the at least one divider is configured to extend within a cross section of the barrel and abut the plunger at a closed configuration of the multiple compartment syringe.

According to some embodiments, there is provided a multiple compartment syringe, including: a first compartment configured to contain a first content, a second compartment configured to contain a second content, a seal disposed between the first and the second compartments, a breaking mechanism of the seal configured such that applying a force on the seal and/or the one or more compartments break the seal enables mixing of the first and second contents, and a plunger configured to allow administration of the first and second contents only upon mixing thereof.

According to some embodiments, the breaking mechanism is independent from movement of the plunger. According to some embodiments, applying pressure on the one or more compartments or causing movement of one of the first and second compartments relative to each other breaks the seal. According to some embodiments, at an unsealed configuration, the first compartment and the second compartment are in fluid communication. According to some embodiments, wherein the plunger includes a safety mechanism configured to prevent a breaking of the seal. According to some embodiments, including at least one more (third) compartment and at least one more (second) seal disposed between the second compartment and the third compartment.

According to some embodiments, the first and second compartments and/or the seal are configured such that applying a force on the seal and/or the one or more compartments breaks the seal without breaking the second seal. According to some embodiments, the second and third compartments and/or the second seal are configured such that the second seal is breakable without breaking the first seal.

According to some embodiments, there is provided a single compartment syringe, the syringe including: a barrel having a proximal end and a distal end and a lumen longitudinally extending between the proximal end and the distal end thereof, wherein the lumen is configured to receive/house a plurality of contents, a plunger sized to fit inside the lumen, and a mechanism having a mixing pusher, a motion converting element, a mixing mechanism, and a locking mechanism, wherein the locking mechanism is configured to lock to position of the plunger within the lumen and wherein the motion converting element is configured to convert linear motion of the mixing pusher into rotary motion of the mixing mechanism, thereby allowing mixing the plurality of contents housed in the syringe while the position of the plunger locked by the locking mechanism.

According to some embodiments, the single compartment syringe may include a motor configured to facilitate mixing the plurality of contents.

According to some embodiments, there is provided a method for inserting content into the multiple compartment syringe, the method including: positioning an opening of an outer portion of a plunger with a compartment of the barrel of a multiple compartment syringe, aligning an opening of an inner portion of a plunger with the opening of the outer portion of the plunger, and inserting content from the lumen of the inner portion through the openings to the compartment.

According to some embodiments, the method further includes misaligning the openings, thereby preventing content from exiting the compartment through the openings. According to some embodiments, the method further includes at least partially overlapping the openings of the inner portion and the outer portion of the plunger. According to some embodiments, the method further includes inserting a content insertion device through a distal opening of the inner portion through a lumen of the inner portion of the plunger. According to some embodiments, the method further includes aligning an opening of the content insertion device with the aligned and/or overlapping openings of the inner portion and the outer portion of the plunger.

In addition to the exemplary aspects and embodiments described above, further aspects and embodiments will become apparent by reference to the figures and by study of the following detailed description.

### BRIEF DESCRIPTION OF THE FIGURES

Some exemplary implementations of the methods and systems of the present disclosure are described with reference to the accompanying drawings. In the drawings, like reference numbers indicate identical or substantially similar elements.

FIG. 1A, FIG. 1B, and FIG. 1C show cross section view schematic illustrations of a multiple compartment syringe, in accordance with some embodiments of the present invention;

FIG. 2A and FIG. 2B show a cross section view and a side view schematic illustration of a multiple compartment syringe, in accordance with some embodiments of the present invention;

FIG. 3 shows a flowchart of functional steps in a method for filling a multiple compartment syringe, in accordance with some embodiments of the present invention;

FIG. 4A and FIG. 4B, which show side view schematic illustrations of a multiple compartment syringe, in accordance with some embodiments of the present invention;

FIG. 5A and FIG. 5B show perspective view schematic illustrations of exemplary seals of a multiple compartment syringe, in accordance with some embodiments of the present invention; and

FIG. 6A and FIG. 6B show perspective view schematic illustrations of an exemplary seal of a multiple compartment syringe, in accordance with some embodiments of the present invention;

FIG. 7A and FIG. 7B show a schematic illustration of a side view and a cross-sectional side view of a syringe, in accordance with some embodiments of the present invention;

FIG. 8A and FIG. 8B show a schematic illustration of a perspective side view of a mixing mechanism of a syringe, in accordance with some embodiments of the present invention;

FIG. 9 shows a schematic illustration of a cross-sectional side view of a syringe at various steps of operation, in accordance with some embodiments of the present invention,

FIG. 10 shows a schematic illustration of a side view of a syringe including an engine, in accordance with some embodiments of the present invention; and

FIG. 11 shows a schematic illustration of a side view of a syringe including an engine, in accordance with some embodiments of the present invention.

### DETAILED DESCRIPTION

According to an aspect of some embodiments of the present invention there is provided a multiple compartment syringe. According to some embodiments, the multiple compartment syringe may be configured to store a plurality of contents within a plurality of compartments of the syringe, such that the contents within different compartments are sealed from one another during storage. According to some embodiments, the multiple compartment syringe may be configured to enable mixing of the contents stored within different compartments by withdrawal of a plunger of the syringe. According to some embodiments, the multiple compartment syringe may be configured such that withdrawing the plunger enables mixing of the contents without ejecting the contents of the syringe out of a needle hub or needle of the syringe, thereby enable the contents to mix prior to injection.

The term “content” may refer to a liquid, powder, gel, or any combination thereof, such as, but not limited to, hyaluronic acid and/or botulinum toxin.

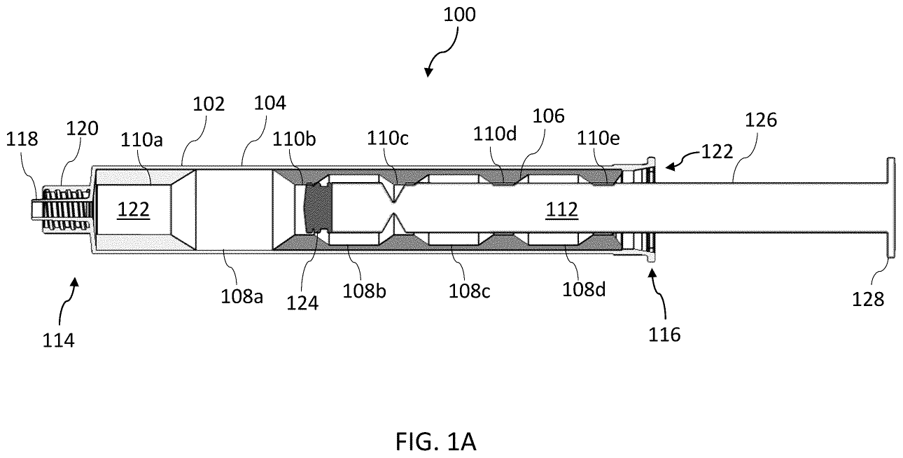

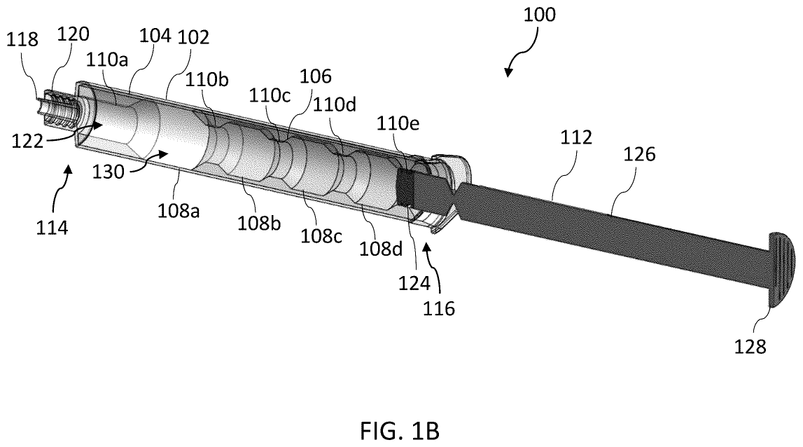

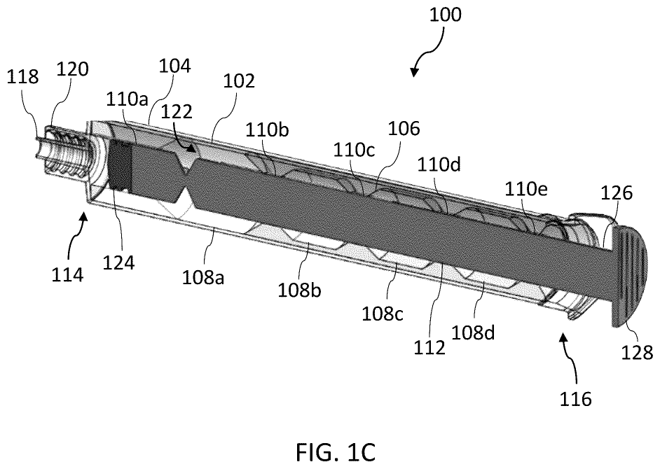

Reference is made to FIG. 1A, FIG. 1B, and FIG. 1C, which show cross section view schematic illustrations of a multiple compartment syringe, in accordance with some embodiments of the present invention.

According to some embodiments, the multiple compartment syringe 100 may include a barrel 102 configured to house one or more contents therein. According to some embodiments, the barrel 102 may include an outer wall 104 having an essentially constant diameter and an inner wall 106 defining two or more compartments 108a/108b/108c/108d (collectively referred to herein as compartments 108). According to some embodiments, the inner wall 106 defines one or more channel 110a/110b/110c/110d/110e (collectively referred to herein as one or more channels 110) which may interconnect between the two or more compartments 108. According to some embodiments, the multiple compartment syringe 100 may include a plunger 112 configured to fit within the one or more channels, such as channels 110b/110c/110d thereby sealing the two or more compartments 108 off from one another. According to some embodiments, the plunger 112 may be sized to fit within the one or more channels 110 such that when the plunger is positioned within the one or more channels 110 the two or more compartments 108 are sealed off from one another, thereby preventing mixing of contents contained in each of the two or more compartments 108, and wherein withdrawal of the plunger 112 from the one or more channels 110 allows mixing of the contents contained in each of the two or more compartments 108.

According to some embodiments, the multiple compartment syringe may be at least partially composed of materials configured to be stored at temperatures that may correspond with storage temperatures of the contents of the compartments 108 and/or optional future contents of the compartments 108. For example, for one or more contents or optionally future contents of the compartments 108 that may require storage at a temperature of below 10 degrees Celsius, for example, such as, botulinum, the multiple compartment syringe may be at least partially composed of materials configured to maintain the structural and functional integrity of the multiple compartment syringe at a temperature of below 10 degrees Celsius. According to some embodiments, the multiple compartment syringe may be at least partially composed of materials which may be inert in relation to the contents or potential future contents of compartments 108, or in other words, the multiple compartment syringe may be composed of materials which may be unreactive with the contents or potential future contents of compartments 108 at the designated storage temperatures of the contents and/or the temperature of the operation in which the syringe and/or the contents or potential future contents of compartments 108 may be used.

According to some embodiments, the barrel 102 may include an elongated member having a distal end 116 and a proximal end 114. According to some embodiments, the barrel 102 may include a lumen 122 extending between the distal end 116 and the proximal end 114. According to some embodiments, and as explained in greater detail elsewhere herein, the lumen 122 may be defined by the inner wall 106 of the barrel 102.

According to some embodiments, the proximal end 114 of the barrel 102 may include a needle hub 118 and/or needle adapter 120 configured to couple to a needle (not shown). According to some embodiments, needle adapter 120 may be interchangeable with one or more other needle adapters, thereby enabling the barrel 102 to couple with one or more different needles which may have different sizes or diameters. According to some embodiments, the lumen 122 may be in fluid communication with the needle adapter 120 and/or needle hub 118, thereby enabling contents to transfer from the lumen 122 of the barrel 102 to through the needle hub 118 at an open configuration of the needle hub 118 and/or needle adapter 120.

According to some embodiments, the lumen 122 may be configured to receive the plunger 112. According to some embodiments, the lumen 122 may extend through the distal end 116 of the barrel 102 such that the plunger may slide through the distal end 116 of the barrel 102. According to some embodiments, the lumen 122 may be defined by the inner wall 106 of the barrel 102.

According to some embodiments, the inner wall 106 of the barrel 102 may vary along the length of the barrel 102, or in other words, the distance between the proximal end 114 and the distal end 116. According to some embodiments, the inner wall 106 may define the plurality of compartments 108. According to some embodiments, the inner wall 106 may define one or more channels 110. According to some embodiments, the one or more channels 110 may be positioned between two consecutive compartments 108. According to some embodiments, the at least one of the one or more channels 110 may be positioned between a compartment 108 and the proximal end 114 of the barrel, such as compartment 108a and channel 110a as depicted in FIG. 1A. According to some embodiments, the at least one of the one or more channels 110 may be positioned between a compartment 108 and the proximal end 116 of the barrel, such as compartment 108d and channel 110e as depicted in FIG. 1A.

According to some embodiments, the inner wall 106 of the barrel 102 at the two or more compartments 108 may have a circumference and/or diameter larger than a circumference and/or diameter of the inner wall 106 at the one or more channels 110. According to some embodiments, the inner wall 106 at the circumference of the one or more channels 110 may be configured to sealingly abut the plunger 112. According to some embodiments, the circumference and/or diameter of the inner wall 106 at the one or more channels 110 may be sized to fit the plunger 112. According to some embodiments, the inner wall 106 at the one or more channels 110 may be configured to sealingly fit the plunger 112, such that a seal is formed between the plunger 112 and the one or more channels 110. Advantageously, the seal formed between the plunger and the one or more channels 110s therefor prevents the contents of one compartment 108 from mixing with contents of a second compartment 108 for when the channel 110 is positioned between these two compartments. FIG. 1 depicts the formation of the seal between the plunger 112 and the channels 110b/110c/110d/110e, thereby preventing contents to flow between the compartments 108a/108b/108c/108d, as well as preventing contents from leaking out of the lumen 122 through the distal end 166 by the seal 110e formed at the distal end 116.

According to some embodiments, one or more portions of the inner wall 106 may include a sealing material, such as a rubber or polymeric substance. According to some embodiments, one or more portions of the inner wall 106 may include a hydrophobic material or a hydrophilic material. According to some embodiments, the one or more portions of the inner wall 106 may include a smooth surface, such as, for example, a surface composed of plastic and/or glass.

According to some embodiments, the plunger 112 may include an elongated body configured to slide through the lumen 122 of the barrel 102. According to some embodiments, the plunger 112 may be moveable in relation to the barrel 102. According to some embodiments, the plunger 112 may be positioned concentrically in relation to the barrel 102. According to some embodiments, the plunger 112 may be positioned eccentrically in relation to the barrel 102. According to some embodiments, the plunger 112 may include a thumb rest 128 configured to aid a user with moving the plunger 112 in relation to the barrel 102. According to some embodiments, the plunger may include a bulb 124 at a proximal end thereof. According to some embodiments, the plunger 112 may comprise one or more of a rigid material, a semi-rigid material, a flexible material, or any combination thereof. According to some embodiments, the plunger 112 may include a rigid frame surrounded by a flexible or compliable material. According to some embodiments, the plunger may include one or more portions surrounded by a material configured to adhere and/or compress against the inner wall 106 at the one or more channels 110.

According to some embodiments, the bulb 124 may be configured to from the same or different material than the body 126 of the plunger 112. According to some embodiments, the bulb 124 may be flush with the body 126 of the plunger 112. According to some embodiments, the bulb may have a larger diameter, or a slightly larger diameter than the body 126 of the plunger 112. According to some embodiments, the bulb 124 and the body 126 of the plunger 112 may be configured to slightly comply under pressure within the one or more channels 110, thereby forming a seal therewith.

According to some embodiments, each one of the one or more compartments 108 may be configured to store individual contents, such as different contents, which may include liquids, powders, and the like. According to some embodiments, different liquid contents may have different viscosities. According to some embodiments, different powder contents may have different particle sizes.

According to some embodiments, at least one of the one or more compartments 108 may include a mixing compartment, such as mixing compartment 108a as depicted in FIG. 1A, FIG. 1B, and FIG. 1C. According to some embodiments, the mixing compartment may remain empty of contents at a closed configuration of the syringe 100. According to some embodiments, the mixing compartment may be configured to receive contents from one or more other compartments 108b/108c/108d at an open configuration of the multiple compartment syringe 100, in which the plunger has been at least partially withdrawn, thereby enabling mixing of two or more contents from two or more compartments. According to some embodiments, the mixing compartment 108a may be positioned at the distal end 114 of the barrel 102. According to some embodiments, the mixing compartment 108a may be in fluid communication with the needle hub 118 and/or needle adapter 120 via one or more channels, such as channel 110a. According to some embodiments, the mixing compartment may be positioned closest to the needle hub and/or distal end of the barrel 102, in relation to the other compartments 108. According to some embodiments, the mixing compartment 108a may be larger than one or more of the other compartments 108, such that contents from the other compartments 108 may fit within the mixing compartment 108a together. According to some embodiments, the syringe may include more than one mixing compartment 108a. According to some embodiments, the mixing compartment may be positioned between two compartments 108.

According to some embodiments, such as depicted in FIG. 1B, at an open configuration of the syringe 100, at least one of the channels 110 is not obstructed or sealed with the plunger 112. According to some embodiments, at an open configuration of the syringe 100, two or more compartments 108 are in fluid communication, or in other words, two or more of the compartments 108 form a single compartment 130.

According to some embodiments, such as depicted in FIG. 1C, at a closed configuration of the syringe 100, at least one of the channels 110 is obstructed and/or sealed by the plunger 112. According to some embodiments, at a closed configuration, space and/or volume of the one or more of the compartments 108 may surround the plunger 112. According to some embodiments, at a closed configuration of the syringe 100, space and/or volume of the one or more of the compartments 108 that surrounds the plunger 112, or at least partially surrounds the plunger 112, may be sealed off from other compartments 108. According to some embodiments, the space and/or volume of the one or more of the compartments 108 that surrounds the plunger 112, or at least partially surrounds the plunger 112, may be sealed off from other compartments 108 by the plunger 112 and one or more of the channels 110 abutting each other.

According to some embodiments, the plunger 112 may include a mixing mechanism configured to stir contents within the one or more compartments 108 at the open configuration of the multiple compartment syringe 100. According to some embodiments, the plunger may include fluted portions configured to increase the movement dynamics of the contents within the one or more compartments. According to some embodiments, the plunger may include leaflets and/or fins coupled thereto, and configured to propel a movement within the one or more compartments 108 during movement of the plunger 112 in relation to the barrel 102 and/or within the one or more compartments 108. According to some embodiments, the plunger 112 may be rotatable in relation to the barrel 102. According to some embodiments, the leaflets and/or fins may be flexible and/or compressible such that when the plunger slides through the one or more channels 110, the leaflets and/or fins may sandwich between the body 126 of the plunger 112 and the inner wall 106 at the one or more channels 110.

According to some embodiments, and as described in greater detail elsewhere herein, the plunger 112 and/or barrel 102 may include a content insertion mechanism configured to enable insertion of content into one or more of the compartments 108 at a closed configuration of the multiple compartment syringe 100. According to some embodiments, the content insertion mechanism may enable a user to insert content into the one or more compartments 108 while the plunger 112 is positioned within the one or more channels 110. According to some embodiments, the content insertion mechanism may enable a user to insert content into the one or more compartments 108 while the syringe 100 is in a closed or semi-open configuration. According to some embodiments, the content insertion mechanism may include a slot or opening configured to enable fluid communication between a lumen of the plunger 112 and the one or more compartments 108.

According to some embodiments, multiple compartment syringe 100 may include a motor. According to some embodiments, the motor may be a mechanical motor. According to some embodiments, distally pushed thumb rest 128 may activate the motor, which, in turn, may activate the mixing mechanism. According to some embodiments, the motor may be configured to facilitate stirring contents within the one or more compartments 108.

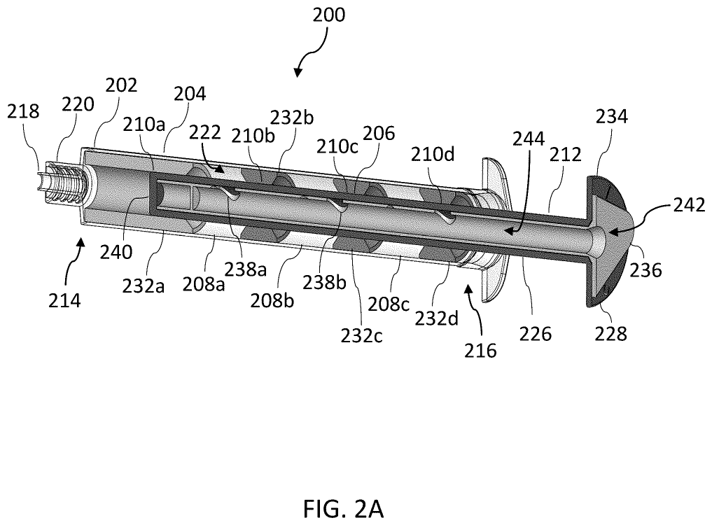

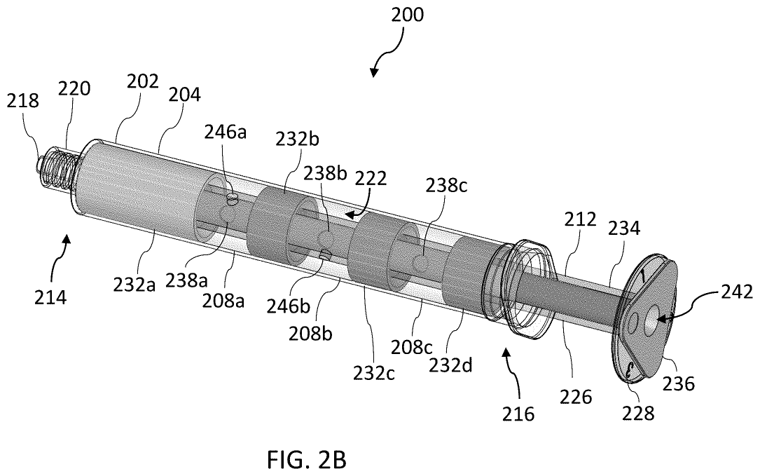

Reference is made to FIG. 2A and FIG. 2B, which show a cross section view and a side view schematic illustration of a multiple compartment syringe, in accordance with some embodiments of the present invention.

According to some embodiments, the multiple compartment syringe 200 as depicted in FIG. 2A and FIG. 2B may be similar to and comprise similar elements as the multiple compartment syringe 100 of FIG. 1A, FIG. 1B, and FIG. 1C.

According to some embodiments, the syringe 200/100 and/or the inner wall 201/106 of the barrel 202/102 may include one or more dividers 232a/232b/232c/232d (collectively referred to herein as one or more dividers 232) configured to divide the lumen 222/122 of the barrel 202/102 into one or more compartments 208/108. According to some embodiments, the one or more dividers 232 may be configured to prevent fluid communication between two or more compartments 208/108. According to some embodiments, at a closed configuration of the syringe 200/100, the one or more dividers 232 are configured to extend within a cross section of the barrel 202/102 and abut the plunger 212/112. According to some embodiments, at a closed position of the syringe 200/100, the one or more dividers 232 may be positioned between the outer wall 204/104 of the barrel 202/102 and the plunger 212/112. According to some embodiments, the one or more dividers 232 may be fixed in relation to the barrel 202/102 or moveable in relation to the barrel 202/102. According to some embodiments, the dividers may define the inner wall 206/106 of the barrel 202/102 at the one or more channels 210/110. According to some embodiments, the one or more channels 110 may be positioned within a lumen of the one or more dividers 232. According to some embodiments, the lumen of the one or more dividers 232 may be sized to sealingly fit the plunger 212/112.

According to some embodiments, one or more portions of the one or more dividers 232 may include a sealing material, such as a rubber or polymeric substance. According to some embodiments, one or more portions of one or more dividers 232 may include a hydrophobic material or a hydrophilic material. According to some embodiments, the one or more portions of the one or more dividers 232 may include a smooth surface, such as, for example, a surface composed of plastic and/or glass.

According to some embodiments, and as described in greater detail elsewhere herein, the plunger 122/112 may include a content insertion mechanism configured to enable insertion of content into one or more of the compartments 208/108 at a closed configuration of the multiple compartment syringe 200/100, thereby enabling a user to insert content into the one or more compartments 208/108 while the compartments are sealed off from one another, and thereby preventing mixing of the inserted contents of different compartments 208/108.

According to some embodiments, the plunger 212/112 may include a lumen and a sealable opening, the lumen enabling insertion of content through the sealable opening into at least one compartment of the barrel 202/102. According to some embodiments, the plunger 122/112 may include an outer portion 234 and an inner portion 236. According to some embodiments, the outer portion 234 and the inner portion 236 may each include one or more openings which form the sealable opening. According to some embodiments, the sealable opening may be moveable in relation to the barrel 202/102 such that at an open position of the sealable opening, the lumen of the plunger 212/112 is in fluid communication with one of the compartments 208/108 of the plurality of compartments 208/108 of the barrel 202/102.

According to some embodiments, the outer portion 234 and the inner portion 236 may be configured to enable insertion of content into one or more of the compartments 208/108. According to some embodiments, the inner portion 236 may fit within the outer portion 234. According to some embodiments, the outer portion 234 and/or the inner portion 236 may include an elongated tubular member, such as a tube or a sheath. According to some embodiments, According to some embodiments, the outer portion 234 and/or the inner portion 236 may include a rigid or semi-rigid material. According to some embodiments, the outer portion 234 may include a sealing material, such as a flexible material, e.g., rubber, configured to sealingly abut the one or more inner channels 210/110. According to some embodiments, the outer portion 234 may include a sealing material about an outer wall thereof.

According to some embodiments, the inner portion 236 may be configured to fit within a lumen of the outer portion 234. According to some embodiments, the inner portion 236 may be configured to slide through and/or in relation to the outer portion 234. According to some embodiments, the inner portion 236 may be configured to rotate within and/or in relation to the outer portion 234. According to some embodiments, the inner portion 236 and the outer portion 234 may be concentric. According to some embodiments, the inner portion 236 may be sized to abut and/or fit within the outer portion 234.

According to some embodiments, the outer portion 234 and the inner portion 236 may be configured to enable insertion of content into one or more of the compartments 208/108 through a distal opening 242 of the inner portion 236. According to some embodiments, the inner portion 236 may extend through a distal opening of the outer portion 234 such that the distal opening 242 and/or a lumen 244 of the inner portion 236 may be exposed to (or in other words, in fluid communication with) the surrounding of the syringe 200/100.

According to some embodiments, the distal opening 242 of the inner portion 236 may be configured to fit a content insertion device, such as, for example, a hose or needle. According to some embodiments, the distal opening 242 of the inner portion 236 may be flush with the lumen of the inner portion 236. According to some embodiments, the plunger 212/112, the inner portion 236, and/or the outer portion 234 may include a barrier configured to prevent content positioned in a lumen of the inner portion 236 from reaching and/or flowing to barrel 202/102, the needle hub 218/118 and/or the needle adapter 220/120, such as, for example, the proximal wall 240, as depicted in FIG. 2A.

According to some embodiments, the outer portion 234 may include one or more openings 246a/246b (collectively referred to herein as openings 246 of the outer portion 234). According to some embodiments, the one or more openings 246 of the outer portion 234 may be positioned along the outer portion 234 at one or more locations corresponding to the one or more compartments 208/108. According to some embodiments, the outer portion 234 may include at least one opening 246 positioned at at least one of the one or more compartments 208/108. According to some embodiments, the outer portion may include at least two openings 246 positioned at at least one of the one or more compartments 208/108. According to some embodiments, the outer portion 234 may include at least one opening 246 positioned (or corresponding to) each compartment. According to some embodiments, the outer portion 234 may include an area corresponding with one or more of the compartments 208/108 that may not include an opening 246, such as, e.g., a compartment which does not need to be filled, for example, such as the mixing compartment 208c/108a.

According to some embodiments, the inner portion 236 may include one or more openings 238a/238b/238c (collectively referred to herein as openings 248 of the inner portion 236). According to some embodiments, the size of the one or more openings 246 of the outer portion 234 may be bigger, smaller, or essentially equal to the size of the openings 248 of the inner portion 236. According to some embodiments, the one or more openings 248 of the inner portion 236 may be configured to overlap with one or more openings 246 of the outer portion 234.

Advantageously, the overlap of an opening 248 of the inner portion 236 and an opening 246 of the outer portion 234 may enable a user to insert content into one or more compartments through the openings 248/246, thereby inserting content from the lumen of the inner portion 236 to the one or more compartments 208/108 or the barrel 202/102. According to some embodiments, overlapping two or more openings 248/246 may be achieved by rotating, sliding, and/or moving the inner portion 236 in relation to the outer portion 234. According to some embodiments, the one or more openings 246/248 of the outer and/or inner portions 234/236 may be positioned at different angles about a longitudinal axis of the plunger 212/112, such that overlapping a first opening 246a of the outer portion 234 with a first opening 238a of the inner portion 236 may result in that other openings 246b/238b/238c may not overlap. According to some embodiments, the inner portion 236 may include only one opening 238 which may require moving, sliding, and/or rotating to overlap with one or more openings 246 or the outer portion 234. According to some embodiments, the syringe 200/100 may include a plug (not shown) configured to fit within the lumen of the inner portion 236, or a lock (lock shown) configured to prevent the inner portion from moving in relation to the outer portion 234 once content is inserted into the one or more compartments 208/108.

According to some embodiments, the outer portion 234 may be rotatable within the barrel 202/102, such that during insertion of content from the lumen of the inner portion 236 through the openings 238/246 and to the one or more compartments 208/108, the rotation may enable to distribute the content throughout the volume of the one or more compartments 208/108 (i.e., around the plunger 212/112 within the lumen 222/122 of the barrel 202/102 within the one or more compartments 208/108).

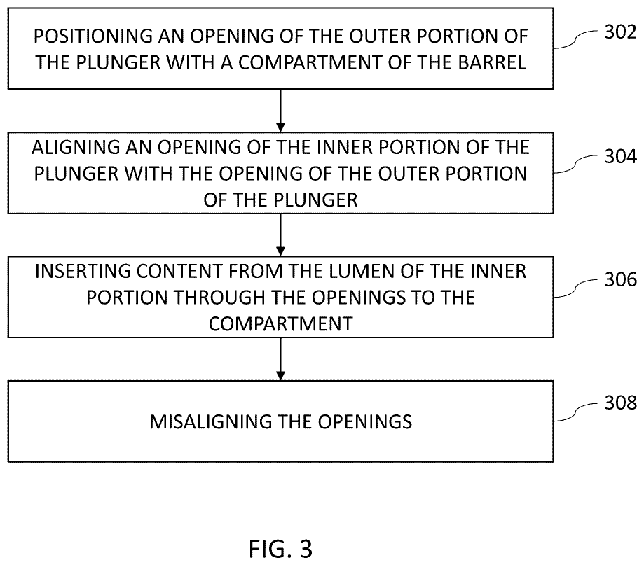

Reference is made to FIG. 3, which shows a flowchart of functional steps in a method for filling a multiple compartment syringe, in accordance with some embodiments of the present invention.

According to some embodiments, there is provided a method for inserting content into the multiple compartment syringe, such as the multiple compartment syringe 200/100. According to some embodiments, at step 302, the method may include positioning an opening of the outer portion of the plunger with a compartment of the barrel. According to some embodiments, at step 304, the method may include aligning an opening of the inner portion of the plunger with the opening of the outer portion of the plunger. According to some embodiments, at step 306, the method may include inserting content from the lumen of the inner portion through the openings to the compartment. According to some embodiments, at step 308, the method may include misaligning the openings, thereby preventing content from exiting the compartment through the openings.

According to some embodiments, at step 302, the method may include positioning an opening of the outer portion of the plunger with a compartment of the barrel. According to some embodiments, the method may include rotating and/or sliding the outer portion of the plunger in relation to the barrel. According to some embodiments, the method may include positioning the opening of the outer portion facing the volume of the compartment. According to some embodiments, at step 304, the method may include aligning an opening of the inner portion of the plunger with the opening of the outer portion of the plunger. According to some embodiments, method may include rotating and/or sliding the inner portion of the plunger in relation to the outer portion of the plunger. According to some embodiments, the method may include at least partially overlapping the openings of the inner portion and the outer portion.

According to some embodiments, at step 306, the method may include inserting content from the lumen of the inner portion through the openings to the compartment. According to some embodiments, the method may include entering content through the lumen of the inner portion of the plunger. According to some embodiments, the method may include entering content through the lumen of the inner portion of the plunger using a content insertion device, such as, for example, a needle, tube and/or hose. According to some embodiments, the method may include entering content through the lumen of the inner portion of the plunger through the distal opening of the inner portion of the plunger. According to some embodiments, the method may include positioning (or aligning) an opening of the content insertion device at the openings of the inner portion and outer portion. According to some embodiments, the method may include ejecting the content from the content insertion device to the compartment through the openings of the inner portion and outer portion.

According to some embodiments, once all the content is ejected into the compartment, the method may include removing the content insertion device from the openings. According to some embodiments, the method may include removing the content insertion device from the lumen of the inner portion of the plunger. According to some embodiments, the method may include misaligning the openings, such that the content cannot escape from the compartment back into the lumen of the inner portion of the plunger.

According to some embodiments, there is provided a method for inserting content into the multiple compartment syringe, such as the multiple compartment syringe 200/100. According to some embodiments, the method may include positioning and/or stacking the one or more dividers at the distal portion of the plunger. According to some embodiments, the method may include inserting a first content into the lumen of the barrel. According to some embodiments, the method may include sliding a first divider towards the distal portion of the plunger, thereby sealingly trapping the first content. According to some embodiments, the method may include inserting a second content into the barrel of the lumen of the barrel. According to some embodiments, the method may include sliding a second divider towards the proximal portion of the plunger, thereby sealingly trapping the second content between the first divider and the second divider.

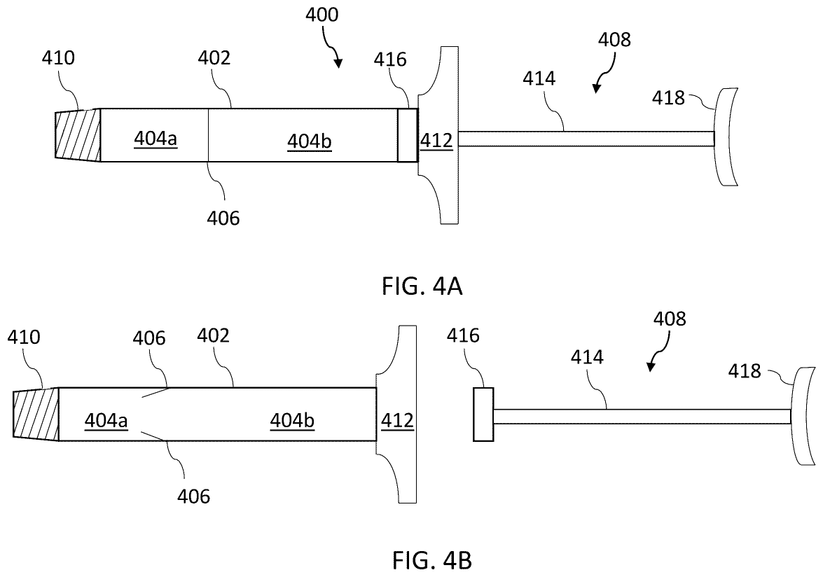

Reference is now made to FIG. 4A and FIG. 4B, which show side view schematic illustrations of a multiple compartment syringe, in accordance with some embodiments of the present invention.

According to some embodiments, the multiple compartment syringe 400/200/100 may include a barrel 402/202/102 configured to contain a plurality of separated contents. According to some embodiments, the multiple compartment syringe 400/200/100 and/or the barrel 402/202/102 may include a plurality of compartments, such as the compartments 404a/404b depicted in FIG. 4A and FIG. 4B, (collectively referred to herein as compartments 404). According to some embodiments, the multiple compartment syringe 400/200/100 and/or the barrel 402/202/102 may include a first compartment 404a configured to contain a first content and a second compartment 404b configured to contain a second content. According to some embodiments, the multiple compartment syringe 400/200/100 and/or the barrel 402/202/102 may include a seal 406 disposed between the first compartment 404a and the second compartment 404b. According to some embodiments, seal 406 is breakable, wherein at an unsealed configuration of the seal 406, the first compartment 404a and the second compartment 404b are in fluid communication. According to some embodiments, the multiple compartment syringe 400/200/100 may include a plunger 408 configured to allow administration of the first and second contents only upon mixing thereof.

According to some embodiments, the barrel 402/202/102 may include a hollow elongated member. According to some embodiments, the barrel 402/202/102 may include a tubular shape, and/or may include a circular transverse cross-section. According to some embodiments, the barrel 402/202/102 may include a lumen configured to contain a plurality of contents. According to some embodiments, the lumen of the barrel 402/202/102 is separated into compartments 404 by one or more seals 406. According to some embodiments, the barrel 402/202/102 may be coupled to a lock tip 410 and/or to a needle hub at a first end of the barrel 402/202/102. According to some embodiments, the lock tip 410 is configured to couple to a needle hub. According to some embodiments, and as described in greater detail elsewhere herein, the barrel 402/202/102 may be configured to couple to a plurality of type of needle hubs and/or needles. According to some embodiments, the barrel 402/202/102 may be coupled to a flange 412 at a second end of the barrel 402/202/102. According to some embodiments, the flange 412 may be configured to enable a user to stabilize the multiple compartment syringe 400/200/100 during use thereof.

According to some embodiments, the barrel 402/202/102 may include the first compartment 404a and the second compartment 404b. According to some embodiments, the first compartment 404a and the second compartment 404b may be configured to contain a first content and a second content, respectively. According to some embodiments, the size of the first compartment 404a and/or the second compartment 404b may vary. According to some embodiments, the size of the first compartment 404a and/or the second compartment 404b may correspond to the amount of the first content and/or the second content. According to some embodiments, the compartments 404 may be sized to fit the contents therein such that at an unsealed configuration of the multiple compartment syringe, such as depicted in FIG. 4B, the barrel 402 may include extra space that is not taken up by the contents, thereby enabling room for mixing of the contents. According to some embodiments, at a sealed configuration of the multiple compartment syringe, such as depicted in FIG. 4A, the first content positioned within first compartment 404a is separated from the second content in the second compartment 404b, or in other words, the first compartment 404a and the second compartment 404b are not in fluid communication.

According to some embodiments, the first compartment 404a and/or the second compartment 404b may be configured such that applying a force onto the first compartment 404a and/or the second compartment 404b may break the seal 406. According to some embodiments, applying a force onto the first compartment 404a and/or the second compartment 404b may include applying positive pressure onto the outer wall of the barrel 402 at the location of the first compartment 404a and/or the second compartment 404b. According to some embodiments, applying a force onto the first compartment 404a and/or the second compartment 404b may include twisting the compartments in relation to another. According to some embodiments, applying pressure on the one or more compartments 404 or causing movement of one of the first compartment 404a and second compartment 404b relative to each other breaks the seal.

According to some embodiments, the wall of the barrel 402/202/102 surrounding the first compartment 404a and/or the second compartment 404b may be configured to succumb to positive pressure. According to some embodiments, the wall of the barrel 402/202/102 surrounding the first compartment 404a and/or the second compartment 404b may be semi-rigid and/or flexible. According to some embodiments, the wall of the barrel 402/202/102 surrounding the first compartment 404a and/or the second compartment 404b may be rigid. According to some embodiments, the wall of the barrel 402/202/102 surrounding the first compartment 404a and/or the second compartment 404b may include one or more semi-rigid and/or flexible regions.

According to some embodiments, the seal 406 may be disposed between each pair of consecutive compartments 404, such as, for example, the first compartment 104a and the second compartment 404b. According to some embodiments, the seal 406 may extend through a cross section of the lumen of the barrel 402/202/102. According to some embodiments, the seal 406 may be configured to seal off a first portion of the lumen of the barrel 402/202/102 from a second portion of the lumen of the barrel 402/202/102. According to some embodiments, and as described in greater detail elsewhere herein, the seal 406 may be configured such that applying a force on the seal 406 breaks the seal 406. According to some embodiments, the seal 406 may include a breaking mechanism configured to break when a force is applied thereto. According to some embodiments, the breaking mechanism may be independent from movement of the plunger. According to some embodiments, the plunger may include a safety mechanism configured to prevent a breaking of the seal 406.

Advantageously, a multiple compartment syringe in which a seal prevents the fluid communication between two compartments, and wherein breaking of the seal allows fluid communication between two compartments, enables a user to mix two products which were initially separated prior to pushing the mixture out of the syringe.

According to some embodiments, the multiple compartment syringe 400/200/100 may include a plunger 408 configured to slide within the barrel 402/202/102. According to some embodiments, the plunger 408 comprises a safety mechanism configured to prevent a breaking of the seal 406. According to some embodiments, the plunger 408 may be configured such that movement of the plunger 408 draws or expels the contents positioned within the barrel 402/202/102. According to some embodiments, the plunger 408 may include a blub 416 at a distal end thereof. According to some embodiments, the bulb 416 may be configured to adhere to the inner wall of the barrel 402/202/102. According to some embodiments, the bulb 416 may be configured to prevent a breaking of the seal 406. According to some embodiments, and as described in greater detail elsewhere herein, the bulb 416 may be configured to fit and/or slide within the barrel 402/202/102 such that, at an unsealed configuration of the syringe, the bulb 416 may adhere to the inner walls of the barrel 402/202/102 until the bulb 416 reaches the broken seal 406. According to some embodiments, the bulb 416 may be configured to slide through the barrel 402/202/102 and past the seal 406 by adhering to a surface of the seal 406 at a broken configuration thereof. According to some embodiments, at a broken configuration of the seal 406, the seal 406 may succumb to the pressure of the bulb 412, thereby adhering to the inner wall of the barrel 402/202/102. According to some embodiments, the bulb 416 may be composed of a flexible or semi-flexible material, such as, for example, rubber. According to some embodiments, the plunger 408 may include a stem 414 coupled to the bulb 416 and configured to slide the bulb within the barrel 402/202/102. According to some embodiments, the plunger 408 may include a stabilizer 418 coupled to the stem and configured to aid a user in stabilizing the movement of the plunger 408 while sliding within the barrel 402/202/102.

According to some embodiments, the multiple compartment syringe 400/200/100 may include at least one more (third) compartment and at least one more (second) seal disposed between the second compartment and the third compartment. According to some embodiments, the first and second compartments and/or the seal may be configured such that applying a force on the seal and/or the one or more compartments breaks the seal without breaking the second seal. According to some embodiments, the second and third compartments and/or the second seal may be configured such that the second seal is breakable without breaking the first seal.

According to some embodiments, the multiple compartment syringe 400/200/100 may include a motor (not shown). According to some embodiments, the motor may be a mechanical motor. According to some embodiments, the motor may be configured to facilitate stirring contents stored within the multiple compartment syringe 400/200/100.

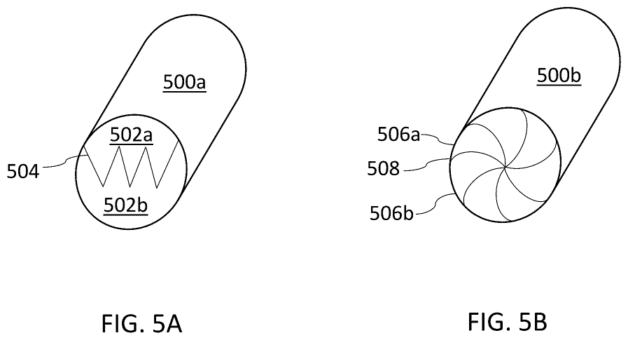

Reference is made to FIG. 5A and FIG. 5B, which show perspective view schematic illustrations of exemplary seals of a multiple compartment syringe, in accordance with some embodiments of the present invention.

According to some embodiments, such as depicted in FIG. 5A and FIG. 5B, the compartments 500a/500b may be sealed by one or more membranes 502a/502b/506a/50b, collectively referred to herein as one or more membranes 502/506). According to some embodiments, the one or more membranes 502/506 may include a thin sheet. According to some embodiments, one or more membranes 502/506 may be configured to seal off the compartment across a cross section thereof. According to some embodiments, one or more membranes 502/506 may be composed of a tearable material, such as plastic, e.g. nylon, which may be configured to tear at designated lines of the seal, such as lines 504/508 of FIG. 5A and FIG. 5B. According to some embodiments, the membranes 502/506 may be configured to tear when pressure is applied onto the compartments 500a/500b.

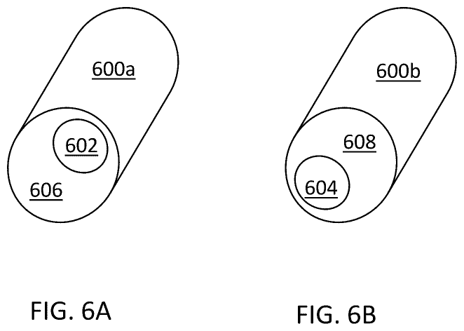

Reference is made to FIG. 6A and FIG. 6B, which show perspective view schematic illustrations of an exemplary seal of a multiple compartment syringe, in accordance with some embodiments of the present invention.

According to some embodiments, such as depicted in FIG. 6A and FIG. 6B, the barrel 402/202/102 may be modular. According to some embodiments, the barrel 402/202/102 may include one or more compartments 600a/600b/208/108. According to some embodiments, the one or more compartments 600a/600b/208/108 may be modular and/or moveable and/or rotatable in relation to each other. According to some embodiments, the one or more compartments 600a/600b/208/108 may include a transverse wall 606/604. According to some embodiments, the transverse wall 606/604 may include one or more openings 602/604.

According to some embodiments, at an open configuration of the syringe, the one or more compartments 600a/600b/208/108 may be positioned such that a transverse wall 606 of a first compartment 600a may seal the opening 604 of the transverse wall 608 of the second compartment 600b. According to some embodiments, at an open configuration of the syringe, the one or more compartments 600a/600b/208/108 may be positioned such that a transverse wall 608 of the second compartment 600b may seal the opening 602 of the transverse wall 606 of the first compartment 600a. According to some embodiments, at an open configuration of the syringe, the one or more compartments 600a/600b/208/108 may be positioned such that the openings 602/604 of the transverse walls 606/604 of the compartments may at least partially overlap, thereby allowing contents from the first compartment 600a to mix with the contents of the second compartment 600b. According to some embodiments, the syringe may be changed into an open configuration from a closed configuration by rotating at least one of the compartments 600a/600b/208/108 in relation to the other.

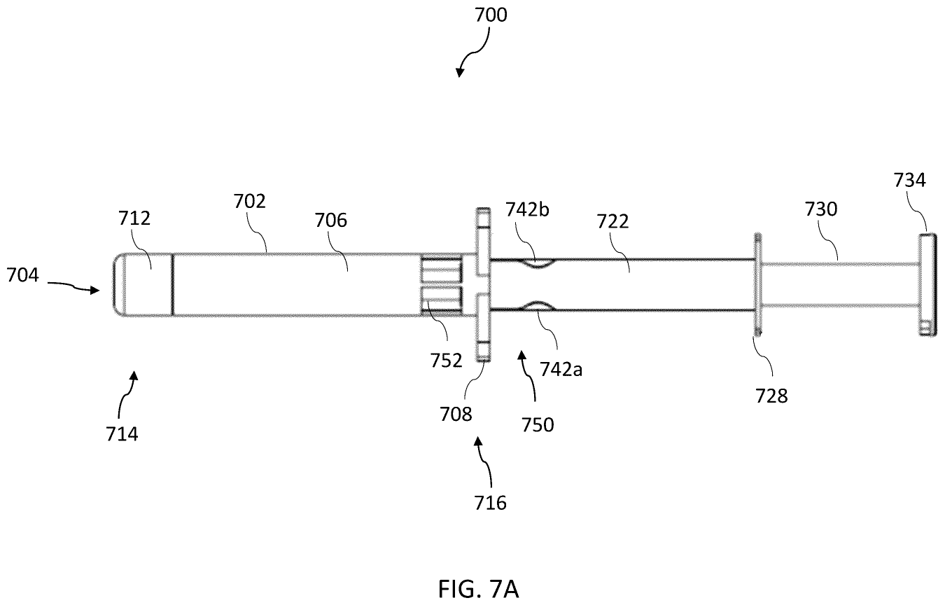

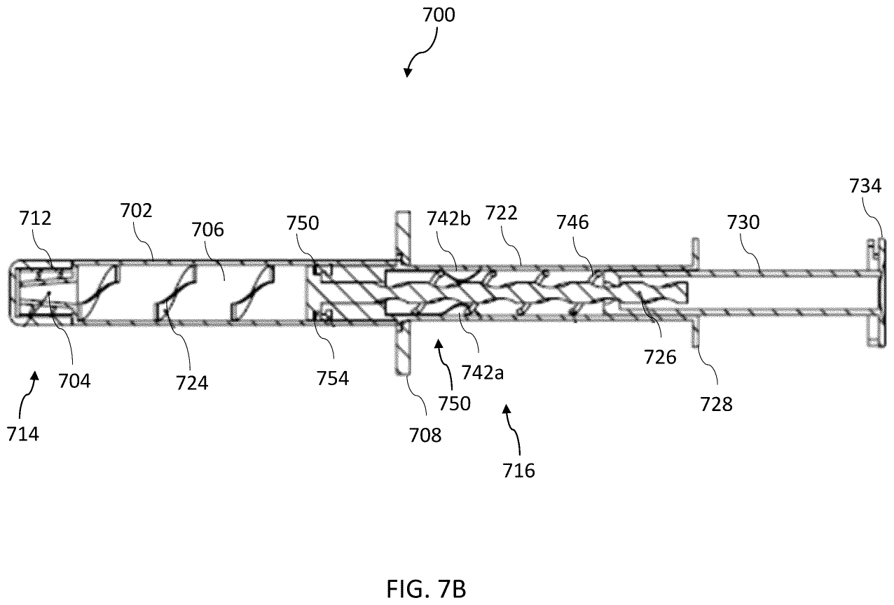

Reference is made to FIG. 7A and FIG. 7B which show a schematic illustration of a side view and a cross-sectional side view of a syringe 700, according to some embodiments.

According to some embodiments, the syringe 700 is configured to enable mixing of a plurality of contents introduced therein. According to some embodiments, the mixing may result in formation of an essentially uniform mixture including the plurality of contents. According to some embodiments, the plurality of contents may include two or more contents, such as liquid contents (e.g., solutions), solid materials (e.g., powders), and the like. As a non-limiting example, the plurality of contents may include a first content in the form of powder and a second content in the form of liquid. According to some embodiments, the plurality of contents may include two or more contents having different viscosity levels. According to some embodiments, the plurality of contents may include two or more powders having different grain sizes. According to some embodiments, the plurality of contents may include hyaluronic acid (HA) and botulinum toxin. According to some embodiments, the HA and/or botulinum toxin may be in a dissolved state. According to some embodiments, the HA and/or botulinum toxin may be in an undissolved state. According to some embodiments, the HA and/or botulinum toxin may be in an undissolved state, such as botulinum toxin powder.

According to some embodiments, the syringe 700 includes a barrel 702 configured to receive/house the plurality of contents therein. According to some embodiments, the barrel 702 includes a lumen 706 extending along the length of barrel 702. According to some embodiments, barrel 702 may include or be associated with a barrel flange 708.

According to some embodiments, a distal end 714 of the barrel 702 includes a distal end opening 704 configured to couple a needle (not shown) to the syringe 700 and to allow introduction of each of the plurality of contents into lumen 706. According to some embodiments, the distal opening 704 may include a needle adapter and/or a needle hub (not shown). According to some embodiments, the needle adapter and/or a needle hub may include a luer lock (e.g., screw connection) tip, slip or push tip/connection, eccentric tip (e.g., used for surface veins and/or artery injections), elongated and/or tapered slip tip, and the like.

According to some embodiments, the distal end 714 may include a cap 712 detachably associated with the distal opening 704 and configured to cover/seal the distal opening 704 when needed.

According to some embodiments, lumen 706 is configured to receive a plunger 722 through the proximal end 716. According to some embodiments, the lumen 706 may be sized to receive the plunger 722.

According to some embodiments, plunger 722 includes an elongated body configured to slide through the distal end of the lumen 706 of barrel 702. According to some embodiments, plunger 722 may be moveable in relation to the barrel 702. According to some embodiments, plunger 722 may be positioned essentially concentrically in relation to barrel 702. According to some embodiments, plunger 722 may be positioned eccentrically in relation to the barrel 702.

According to some embodiments, plunger 722 is detachably associated or integrally formed with a plunger flange 728 extending circumferentially therefrom (i.e. perpendicularly to a longitudinal axis of the barrel 702). According to some embodiments, when the plurality of contents is introduced into syringe 700, plunger flange 728 is pulled proximately (i.e., outside lumen 706). According to some embodiments, when the plurality of contents is transferred from syringe 700, plunger flange 728 is pushed distally (i.e., into lumen 706).

According to some embodiments, plunger 722 may include a snap mechanism 750, configured to lock the position of plunger 722 relative to barrel 702. According to some embodiments, plunger 722 may further include plunger releasing buttons 742a/742b configured release snap mechanism 750 thereby releasing plunger 722 from its locked position. According to some embodiments, in the locked configuration, plunger 722 may be retained in a fully extended configuration thereof (e.g., as depicted in FIG. 7A). According to some embodiments, in the locked configuration, plunger 722 may be retained in a partially extended configuration thereof (e.g., as depicted in FIG. 7A). According to some embodiments, in the released configuration, plunger 722 may be distally pushed (e.g., by a user) for transferring the plurality of contents from/into the syringe 700, as explained in greater detail elsewhere herein. According to some embodiments, snap mechanism 750 may include one or more grooves 752 configured to receive snap mechanism 750, thereby locking the position of the plunger 722.

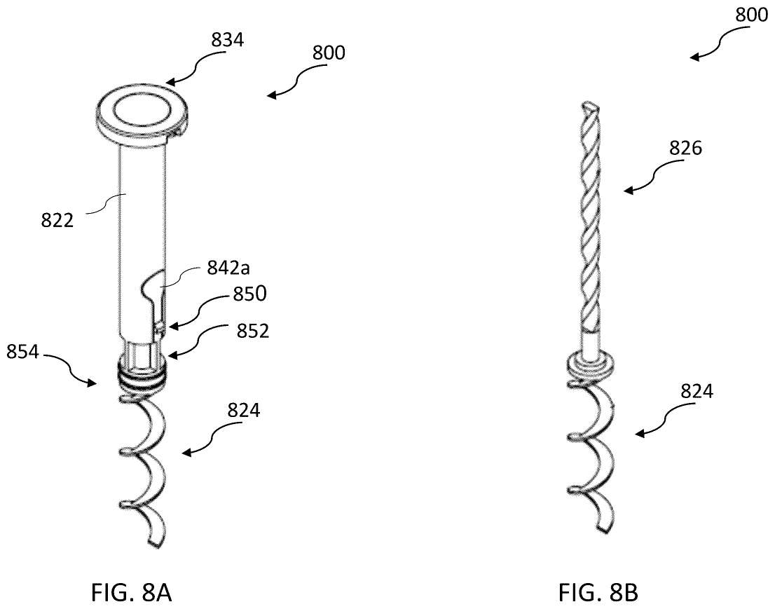

According to some embodiments, plunger 722 includes a mixing mechanism 724 configured to mix/stir the plurality of contents retained within the barrel 702, resulting in formation of an essentially uniform mixture, as explained in greater detail with reference to FIGS. 8A and 8B.

According to some embodiments, plunger 722 is associated with a mixing pusher 730. According to some embodiments, mixing pusher 730 is configured to allow a user to controllably activate mixing mechanism 724 of plunger 722, thereby mixing the plurality of contents stored in barrel 702. According to some embodiments, mixing pusher 730 includes a mixing pusher flange 734, configured to aid a user with moving the mixing pusher 730.

According to some embodiments, plunger 722 includes a return mechanism 746. According to some embodiments, return mechanism 746 is configured to facilitate the activation of mixing mechanism 724. According to some embodiments, upon distally pushing the mixing pusher flange 734 (i.e., applying linear force downwards) in order to initiate mixing of the plurality of contents (i,e., while the positions of plunger 722 and plunger flange 728 are fixed), mixing pusher 730 is pushed distally, applying a force on return mechanism 746. Consequently, a restoring force is exerted by return mechanism 746 (i.e., a force in an opposite direction), thereby proximately pulling mixing pusher flange 734 back to the initial position thereof. Put differently, due to return mechanism 746, mixing pusher flange 734 automatically returns to the initial position thereof (i.e., proximately extended position of mixing pusher 730, as depicted, for example, in FIG. 7B), allowing the user repeatedly push mixing pusher flange 734 and hence repeatedly active mixing mechanism 724. According to some embodiments, return mechanism 746 may include a spring.

According to some embodiments, mixing mechanism 724 of plunger 722 includes a motion converting element 726 configured to convert linear motion to rotary motion. According to some embodiments, motion converting element 726 is operably coupled to mixing mechanism 724. According to some embodiments, the mixing mechanism 724 may include, among others, an Archimedes' screw, positioned within the lumen 706. According to some embodiments, upon distally pushing the mixing pusher flange 734 (i.e., applying linear motion downwards), motion converting element 726 is also pushed downwards thereby initiating rotational movement of mixing mechanism 724, resulting in mixing the plurality of contents in the barrel 702 by the rotary motion of the mixing mechanism 724. According to some embodiments, the position of plunger 722 remains fixed during activation of the mixing mechanism 724.

According to some embodiments, plunger 722 may include one or more gaskets 754 configured to seal off a space between the plunger 722 and barrel 702 while allowing rotation of motion converting element 726.

According to some embodiments, the syringe 700 may be an unfilled syringe, allowing a user to separately introduce each of the plurality of contents into the syringe 700.

According to some embodiments, syringe 700 may be partially pre-filled, i.e., filled with a first portion of the plurality of contents, such that in operation, a user will introduce a second portion of the plurality of contents into syringe 700. According to some embodiments, syringe 700 may house a powder (e.g., botulinum toxin) within barrel 702, such that in operation, the user introduces therein HA and mixes the powder and the HA. In particular, in some embodiments, the mixing mechanism 724 may be coated with the powder, thereby housing the powder within barrel 702. According to some embodiments, the inner wall of barrel 702 may be coated with the powder.

According to some embodiments, syringe 700 may be a pre-filled syringe, i.e., configured to store the plurality of contents in the unmixed form thereof. According to some embodiments, barrel 702 may include one or more membranes (not shown) configured to retain each of the plurality of contents in an essentially unmixed form thereof. According to some embodiments, applying pressure in mixing pusher flange 734 may result in imparting pressure on the one or more membranes, thereby initiating mixing the plurality of contents. According to some embodiments, applying pressure of the one or more membranes may result in dislocating of the one or more membranes. According to some embodiments, the one or more membranes may minimize mixing of the plurality of contents by serving as a physical barrier. Alternatively or additionally, in some embodiments, the one or more membranes may include selectivity properties, such that applying pressure thereon will modify the selectivity properties thereof, thereby allowing mixing the plurality of contents. According to some embodiments, the one or more membranes may be flexible.

According to some embodiments, the pre-filled syringe may separately house each of the plurality of contents without mixing thereof by Reference is made to FIG. 8A and FIG. 8B which show a schematic illustration of a perspective side view of a mixing mechanism 800 of a syringe, according to some embodiments.

According to some embodiments, mixing mechanism 800 may correspond to and have a similar structure to the mixing mechanism 724 of syringe 700.

According to some embodiments, the following components depicted in FIG. 8A and FIG. 8B 822, 824, 826, 834, 842a, 850, 854 correspond to and may have the same structure, configuration and/or composition as the previously described components 722, 724, 726, 734, 742a, 750 and 754 in FIGS. 7A and 7B.

According to some embodiments, mixing mechanism 800 is configured to be installed within a syringe (e.g., the syringe 700). According to some embodiments, mixing mechanism 800 can advantageously be operated single handedly by a user by placing the thumb of the user on a mixing pusher flange 834 and pushing thereof.

According to some embodiments, the mixing pusher flange 834 may be repeatedly pushed. According to some embodiments, the mixing pusher flange 834 may be pushed 1, 2, 3, 4, or 5 times to mix the plurality of contents. Each possibility is a separate embodiment.

According to some embodiments, mixing mechanism 800 is configured to convert linear motion (applied by the user on the mixing pusher flange 834) to rotational motion of a mixing mechanism 824. According to some embodiments, each push applied to the mixing pusher flange 834 leads to a predetermined number of rotations of mixing mechanism 824. As a non-limiting example, distally pushed pusher flange 834 causes three rotations of the mixing mechanism 824.

According to some embodiments, mixing mechanism 800 includes a snap mechanism 850 configured to lock/retain the position of a plunger 822. According to some embodiments, in an open configuration of snap mechanism 850, plunger 822 may linearly move (e.g., distally and proximately), e.g., in order to transfer the plurality of contents from/into the syringe. According to some embodiments, in a closed configuration of the snap mechanism 850, the position of the plunger 822 is fixed, such that pushing the mixing pusher flange 834 distally will convert the linear motion thereof to rotational motion of the mixing mechanism 824, thereby mixing the plurality of contents housed in the syringe. According to some embodiments, the snap mechanism 850 may include one or more grooves 852 configured to receive snap mechanism 850, thereby locking the position of the plunger 822.

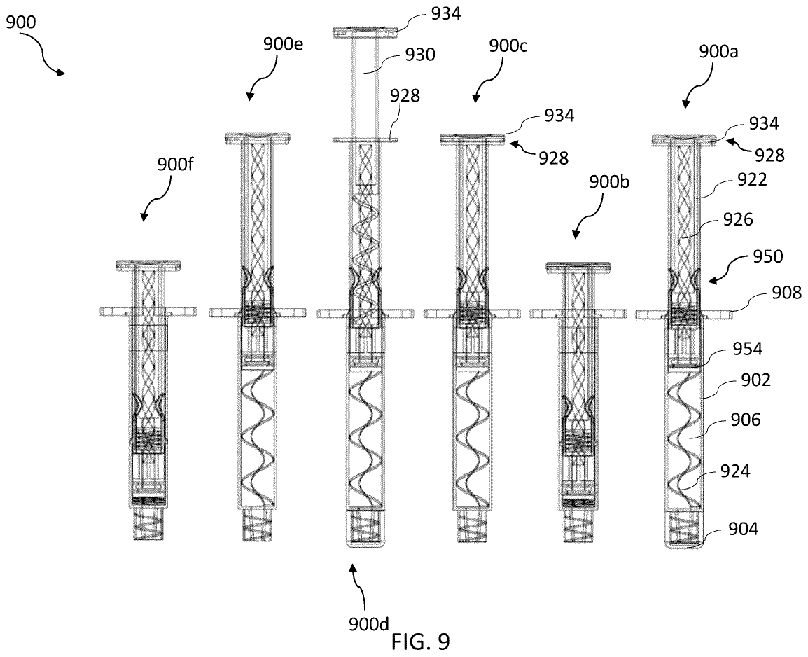

Reference is now made to FIG. 9, which shows a schematic illustration of a cross-sectional side view of a syringe 900 at various steps of operation, according to some embodiments.

According to some embodiments, the following components depicted in FIG. 9 900, 902, 904, 906, 908, 922, 924, 926, 928, 930, 934, 950 and 954 correspond to and may have the same structure, configuration and/or composition as the previously described components 700, 702, 704, 706, 708, 722, 724, 726, 728, 730, 734, 750 and 754 in FIGS. 7A and 7B.

According to some embodiments, in step 900a, the syringe 900 may be an empty (as-received) syringe. According to some embodiments, in the as-received configuration, at least a majority of plunger 922 may be positioned outside a barrel 902. According to some embodiments, in step 900a, the position of a mixing pusher flange 934 is locked to the position of a plunger flange 928.

According to some embodiments, in step 900b, plunger 922 may be pushed distally (i.e. downwards) and slide into a lumen 906 of the barrel 902. According to some embodiments, plunger 922 may be manually pushed by a user. According to some embodiments, plunger 922 may be automatically pushed. According to some embodiments, in step 900b, the position of mixing pusher flange 934 is locked to the position of plunger flange 928.

According to some embodiments, in step 900c, plunger 922 may be proximately pulled (i.e., upwards), resulting in exiting at least a portion of the plunger 922 outside the lumen 906, while transferring a plurality of contents through a distal opening 904 into the syringe 900. According to some embodiments, in step 900c at least a portion of the syringe 900 is filled with the plurality of contents. According to some embodiments, in step 900c, the position of mixing pusher flange 934 is locked to the position of plunger flange 928.

According to some embodiments, in step 900d, a snap mechanism 950 locks the position of the plunger 922, and mixing pusher flange 934 is pushed proximally, thereby releasing a mixing pusher 930. Put differently, mixing pusher 930 is displaced and positioned outside plunger 722. According to some embodiments, distally pushing the mixing pusher flange 934 leads to pushing of the mixing pusher 930, thereby activating motion converting element 926 of syringe 900, which in turn results in rotation of a mixing mechanism 924 and mixing the plurality of contents.

According to some embodiments, upon pushing the mixing pusher flange 934 distally, the mixing pusher 930 may be pushed proximately by motion converting element 934. Put differently, in some embodiments, mixing pusher flange 934 may automatically return to an open configuration thereof (wherein at least a portion of the mixing pusher 930 is positioned outside the plunger 922). According to some embodiments, upon pushing the mixing pusher flange 934 distally to mix the plurality of contents, the position of plunger 922 is fixed.

According to some embodiments, each push applied to the mixing pusher 930 leads to a predetermined number of rotations of mixing mechanism 924. According to some embodiments, a single push of the mixing pusher 930 may lead to 1, 2, 3, 4, 5, or more rotations of the mixing mechanism 924. Each possibility is a separate embodiment.

According to some embodiments, mixing pusher 930 may be repeatedly pushed until achieving an essentially uniform mixture of the plurality of contents.

According to some embodiments, in step 900e, the mixing pusher 930 is pushed distally, and the snap mechanism is shifted into an open configuration thereof, releasing the locked position of the plunger 922. According to some embodiments, in step 900e, the position of mixing pusher flange 934 is locked to the position of plunger flange 928.

According to some embodiments, in step 900f, the plunger is pushed distally, for administrating the uniform mixture from the syringe via distal end 904 of syringe 900.

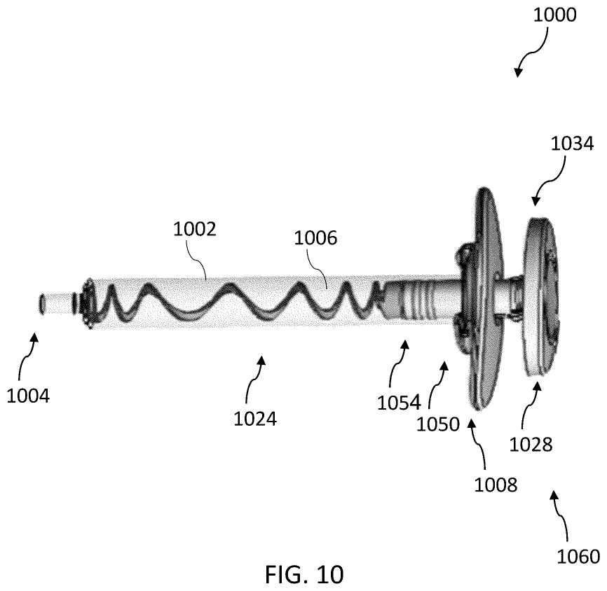

Reference is now made to FIG. 10, which shows a schematic illustration of a side view of a syringe 1000, according to some embodiments.

According to some embodiments, the following components depicted in FIG. 10 1000, 1002, 1004, 1006, 1008, 1024, 1028, 1034, 1050 and 1054 correspond to and may have the same structure, configuration and/or composition as the previously described components 900, 902, 904, 906, 908, 924, 928, 934, 950 and 954 in FIG. 9.

According to some embodiments, syringe 1000 includes a motor 1060. According to some embodiments, motor 1060 may be a mechanical motor. According to some embodiments, motor 1060 may be positioned within a pusher flange 1034. According to some embodiments, motor 1060 may be positioned within a barrel flange 1008.

According to some embodiments, motor 1060 is configured to facilitate mixing a plurality of contents contained in a lumen 1006. According to some embodiments, motor 1060 may include one or more wires. According to some embodiments, the one or more wires may include two wires. According to some embodiments, the one or more wires may be positioned within pusher flange 1034, pass through a plunger 1022 and into a mixing mechanism 1024, thereby facilitating mixing the plurality of contents in lumen 1006.

According to some embodiments, pushing (e.g., by a user) pusher flange 1034 (i.e., into lumen 1006) may activate motor 1060, which, in turn, activates mixing mechanism 1024 by rotating thereof. According to some embodiments, distally pushed pusher flange 1034 may active motor 1060, resulting in activating mixing mechanism 1024, so that a predetermined number of rotations are performed by mixing mechanism 1024.

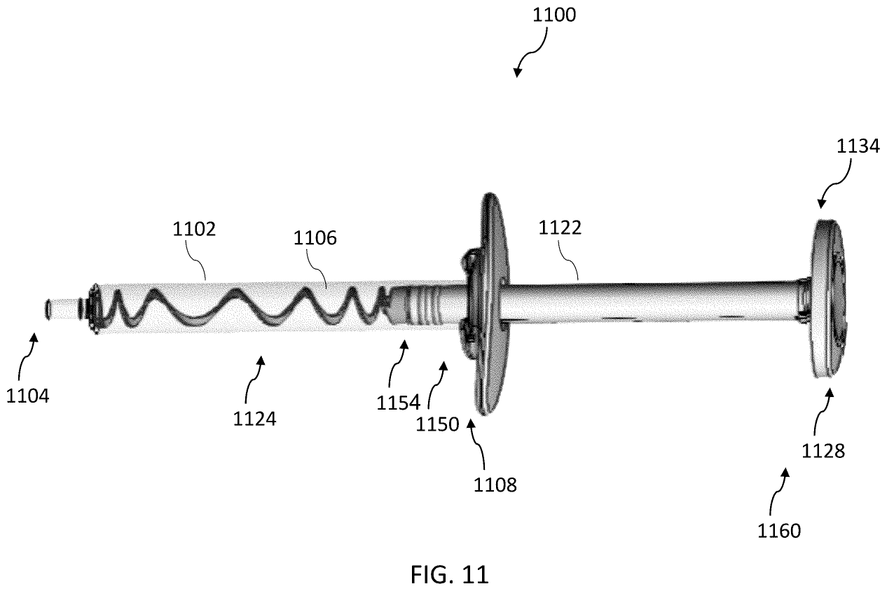

Reference is now made to FIG. 11, which shows a schematic illustration of a side view of a syringe 1100, according to some embodiments.

According to some embodiments, the following components depicted in FIG. 11 1100, 1102, 1104, 1106, 1108, 1122, 1124, 1128, 1134, 1150 and 1154 correspond to and may have the same structure, configuration and/or composition as the previously described components 900, 902, 904, 906, 908, 922, 924, 928, 934, 950 and 954 in FIG. 9.

According to some embodiments, syringe 1100 includes a motor 1160. According to some embodiments, motor 1160 may be a mechanical motor. According to some embodiments, motor 1160 may be positioned within a pusher flange 1134. Alternatively, or additionally, in some embodiments, motor 1160 may be positioned within a barrel flange 1108.

According to some embodiments, motor 1160 is configured to facilitate mixing a plurality of contents contained in a lumen 1106. According to some embodiments, motor 1160 is configured to facilitate administrating the plurality of contents from syringe 1100. According to some embodiments, motor 1160 may include one or more wires. According to some embodiments, the one or more wires may include two wires. According to some embodiments, the one or more wires may be positioned within pusher flange 1134, pass through a plunger 1122 and into a mixing mechanism 1124, thereby facilitating mixing the contents in lumen 1106.

According to some embodiments, pushing (e.g., by a user) pusher flange 1134 (i.e., into lumen 1106) may activate motor 1160, which, in turn, activates mixing mechanism 1124 by rotating thereof. According to some embodiments, distally pushed pusher flange 1134 may active motor 1160, resulting in activating mixing mechanism 1124, so that a predetermined number of rotations are performed by mixing mechanism 1124.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1.-25. (canceled)

26. A multiple compartment syringe, comprising:
a barrel comprising an outer wall having an essentially constant diameter and an inner wall defining at least two compartments and at least one channel interconnecting the at least two compartments; wherein the diameter of each of the at least two compartments is larger than a diameter of the channel, and
a plunger sized to fit within the channel wherein when the plunger is positioned within the channel the at least two compartments are sealed off from one another, thereby preventing mixing of contents contained in each of the at least two compartments; and
wherein withdrawal of the plunger from the channel allows mixing of the contents contained in each of the at least two compartments.

27. The multiple compartment syringe according to claim 26, wherein at least one compartment is a mixing compartment configured to receive contents from a first compartment and a second compartments at an open configuration of the multiple compartment syringe in which the plunger has been at least partially withdrawn, thereby enabling mixing of two or more contents of two or more compartments.

28. The multiple compartment syringe according to claim 26, wherein the plunger comprises a content insertion mechanism configured to enable insertion of content into a compartment at a closed configuration of the multiple compartment syringe in which the plunger is positioned within the channel.

29. The multiple compartment syringe according to claim 28, wherein the plunger comprises a lumen and a sealable opening, the lumen enabling insertion of content through the sealable opening into at least one compartment of the barrel.

30. The multiple compartment syringe according to claim 26, wherein the inner wall of the barrel comprises at least one divider positioned within the lumen of the barrel and configured to prevent fluid communication between two or more compartments.

31. A multiple compartment syringe, comprising:
a first compartment configured to contain a first content;
a second compartment configured to contain a second content;
a seal disposed between the first and the second compartments;
a breaking mechanism of the seal configured such that applying a force on the seal and/or the one or more compartments break the seal enables mixing of the first and second contents; and
a plunger configured to allow administration of the first and second contents only upon mixing thereof.

32. The multiple compartment syringe of claim 31, wherein the breaking mechanism is independent from movement of the plunger, and/or wherein applying pressure on the one or more compartments or causing movement of one of the first and second compartments relative to each other breaks the seal.

33. The multiple compartment syringe of claim 31, wherein at an unsealed configuration, the first compartment and the second compartment are in fluid communication.

34. The multiple compartment syringe of claim 31, wherein the plunger comprises a safety mechanism configured to prevent a breaking of the seal.

35. The multiple compartment syringe of claim 31, further comprising at least one more (third) compartment and at least one more (second) seal disposed between the second compartment and the third compartment.

36. The multiple compartment syringe of claim 35, wherein the second and third compartments and/or the second seal are configured such that the second seal is breakable without breaking the first seal.

37. A syringe, comprising:
a barrel comprising a proximal end and a distal end, and a lumen longitudinally extending between the proximal end and the distal end thereof, wherein the lumen is configured to receive/house a plurality of contents,
a plunger sized to fit inside the lumen, and
a mechanism comprising a mixing pusher, a motion converting element, a mixing mechanism, and a locking mechanism;
wherein the locking mechanism is configured to lock a position of the plunger within the lumen and wherein the motion converting element is configured to convert linear motion of the mixing pusher into rotary motion of the mixing mechanism, thereby allowing mixing the plurality of contents housed in the syringe while the position of the plunger is locked by the locking mechanism.

38. The syringe of claim 37, further comprising a motor configured to facilitate mixing the plurality of contents.

39. The syringe of claim 37, wherein the syringe is a single compartment syringe.

40. The syringe of claim 37, wherein the mixing pusher comprises a mixing pusher flange and the plunger comprises a plunger flange.

41. The syringe of claim 40, wherein the mixing pusher flange is capable of being locked to the plunger flange.

42. The syringe of claim 40, wherein when the mixing pusher flange is moved proximally, the mixing pusher is released from the plunger, while said plunger is locked into position by a snap mechanism.

43. The syringe of claim 40, wherein when the mixing pusher flange is pushed distally, the mixing pusher is pushed, thereby activating the motion converting element.

44. The syringe of claim 40, wherein the mixing pusher flange is configured to return automatically to an open configuration thereof, wherein at least a portion of the mixing pusher is positioned outside the plunger.

45. The syringe of claim 37, wherein each push applied to the mixing pusher leads to a predetermined number of rotations of the mixing mechanism.
